(19) United States
(12) Patent Application Publication
(10) Pub. No.: US 2019/0272913 A1
(43) Pub. Date: Sep. 5, 2019

(54) Method And System For Determining Homeopathic Medicinal Potency To Patient Based On Nigam's Potency Scale

(71) Applicants: Harsh Nigam, Kanpur (IN); Shushant Nigam, Kanpur (IN); Archit Mehrotra, Kanpur (IN)

(72) Inventors: Harsh Nigam, Kanpur (IN); Shushant Nigam, Kanpur (IN); Archit Mehrotra, Kanpur (IN)

(21) Appl. No.: 16/348,875

(22) PCT Filed: Jan. 8, 2018
(86) PCT No.: PCT/IN2018/050010

§ 371 Date: May 10, 2019

(30) Foreign Application Priority Data
Feb. 14, 2017 (IN) 201711005228

(51) Int. Cl. G16H 20/90; G16H 50/20

(52) U.S. Cl. CPC G16H 20/90; G16H 50/20

(57) ABSTRACT

The invention relates to a computer implemented method and system for determining homoeopathic medicinal potency to patient based on Nigam's Potency scale. The said method to determine the homoeopathic medicinal potency based on Nigam's potency scale which is stored in a memory unit which is being retrieved based on the input given to the system through an input device. All instruction are carried out by the processing element. Nigam's potency scale works-in a new way to determine potency. The system takes in a set of numbers depending on the tabs a user clicks, adds them up and displays the result according to the range in which the number falls. The scores range from 18(lowest) to 54(highest). The division of scores and result is done as follows: 1) 18-30: Use low potency (1X/D1 to 6X/D6/3C) 2) 31-42: Use medium potency (4C TO 12C) 3) 43-46: Use high potency (30C) 4) 47-50: Use very high potency (200C) 5) 51-54: Use ultra-high potency (1M, 10M, 50M, 100M/CM; 1M=1000).

### FIELD OF INVENTION

The present invention relates to a computer implemented method and system for determining homoeopathic medicinal potency to patient based on Nigam's Potency scale. The said method to determine the homoeopathic medicinal potency based on Nigam's potency scale which is stored in a memory unit which is being retrieved based on the input given to the system through an input device. All instruction are carried out by the processing element. Nigam's potency scale works-in a new way to determine potency. The system takes in a set of numbers depending on the tabs a user clicks, adds them up and displays the result according to the range in which the number falls.

The scores range from 18 (lowest) to 54 (highest).

The division of scores and result is done as follows:
1) 18-30: Use low potency (1X/D1 to 6X/D6/3C)
2) 31-42: Use medium potency (4C TO 12C)
3) 43-46: Use high potency (30C)
4) 47-50: Use very high potency (200C)
5) 51-54: Use ultra-high potency (1M, 10M, 50M, 100M/CM; 1M=1000)

The judgment is done based on three conditions:
1) Nature of medicine
2) Nature of disease
3) Susceptibility of patient

### BACKGROUND OF THE INVENTION

The Potency

The term Posology originates from Greek words “posos” meaning how much and “logos” meaning study. But the question of posology in Homoeopathic context extends far beyond how much. In Homoeopathy, Homoeopathic dose implies with the potency, quantity and form of medicine and the repetition of the medicine. There are three types of scales used to prepare potencies; the Decimal scale (denoted as D or X Scale where the medicinal substance is diluted at a ratio of 1:10), Centesimal scale (denoted as C Scale where the medicinal substance is diluted at a ratio of 1:100), and 50 Millesimal scale (denoted as LM Scale where the medicinal substance is diluted at a ratio of 1:50000),

The potency are broadly classified into 5 types:
1) Low potency (1X/D1 to 6X/D6/3C)
2) Medium potency (4C TO 12C)
3) High potency (30C)
4) Very high potency (200C)
5) Ultra-high potency (1M, 10M, 50M, 100M/CM; 1M=1000)

As regards potency to be administered to the patient there is as such no fixed rule. It is the matter of experience and the observation. Throughout the Organon we are puzzled with several key questions like what potency, how much, how to administer and to repeat or not to repeat the dose. We do not get the clear idea what Hahnemann means with small or large dose, does he mean the amount or the potency.

This confusion arises because we do not understand German language, Hahnemann in connection to dose uses two words in the Organon Gabe (which is German for giving) and Dosis (which in Greek for giving) both mean a giving and it is the context in which two words are used in the Organon which establishes why he uses two terms. The term Gabe establishes the potency and Dosis the amount.

Dose is defined as that which is given or administered. Dose refers to the amount of a medicinal substance that is administered. This includes both the medicines potency and the amount of medicine given.

Since higher potencies have less material and therefore considered as smaller doses. Small dose in the Organon is referred to a small quantity of a crude substance.

So the question of posology has four elements,

1—What potency?

2—What amount?

3—How to administer the medicine?

4—To repeat or not to repeat?

The Potency Problem

There is no official definition of what the word “potency” means in homeopathy. It is usually used to refer to the degree of dilution that a homeopathic remedy has undergone in its manufacturing process.

It can be elaborated with respect to the administration of suitable potency and come to the individual who is to be administered the potency in the following manner—

1—From Homoeopathic point of view we should understand the individual in regard to the typology, psychological constitution and the biological constitution and also we should know about the diathesis, miasma and the hereditary characteristics.

2—Three factors at play in Homoeopathic curative medicine:

A—Disease

B—Individual

C—Medicine

According to homoeopathic principles all medicines elicit two responses on an individual:

i) The Primary response that is the effect of medicine on the system.

ii) The Secondary response that is the response to the medicine by the system. This secondary response is said to be curative. The curative response is dependent upon the power of stimulus/medicine to elicit a response and the initial state of the system which is responding to the stimulus/medicine.

This initial state of the system (=individual) in homoeopathic literature is known as susceptibility of the patient.

3—The initial state can be judged by the following

a) Typology

b) Biological constitution

c) Psychological constitution

Note: Susceptibility is the general capability of the organism to receive impression or the power of the organism to react to stimuli. The cure of diseases depends upon the same power of the organism to react to the impression of the curative remedy.

Susceptibility of the patient is dependent on the following:

i—Make up of an individual

ii—The type and stage of the disease

The judgment is done based on three conditions:

A—Nature of medicine

B—Nature of Disease

C—Susceptibility of patient

Potency and Susceptibility

1—The selection of potency depends upon the susceptibility of the patient.

2—The higher the susceptibility the higher the potency required to treat the patient.

3—The susceptibility of the patient depends upon the age, sex, mode of living, nature of the disease and stage of the disease, occupation, physical and mental reaction of the individual to various stimuli.

4—It is said that we should start with the medium potencies and then go up to the higher or lower potencies according to the prognosis of the case.

5—Lower potencies should be administered in the patients with structural and organic changes as the susceptibility of these patients is low. Giving high potencies in such cases can produce unnecessary aggravations and may result in decline of the patient.

6—In case of children and young adults higher potencies can be administered as the susceptibility in them is high.

7—The old people or the patients having feeble circulation or in the comatose patients, shock, severe dehydration, severe haemorrhage we should go with low to medium potencies, as administration of high potencies in these cases can lead to prolonged aggravations or sudden death.

8—In chronic diseases the susceptibility becomes low because of long continued suffering, they require low or medium potencies.

9—Persons who are having high intellect for example Doctors, Lawyers, Ministers etc generally require high potencies because of higher susceptibility.

10—The persons who are involved in physical labour generally require lower or medium potencies because of their low susceptibility.

Potency and Plane of Disease

The potency of medicine to be given is to be decided on the basis of plane of disease.

1—If the disease of an individual is confined to body matrix/organic plane; the case should be treated with low potencies.

2—If the disease is in the dynamic plane, the medium potency are to be used.

3—If the disease is confined to the central controlling axis, the case have to be managed with high potencies.

4—If the disease is at mental plane or if the mental symptoms are prominent, we should treat the case with very high potencies

Potency and Physical Constitution

There are three types of physical constitutions:

1—Phosphoric constitution

2—Flouric constitution

3—Carbonic constitution

Phosphoric constitution requires lower potencies. On the other hand flouric constitutions require medium potencies and the carbonic constitution are sluggish and hence require higher potencies.

Potency and Biological Constitution

The biological constitutions are of three types:

1—Carbonitrogenoid constitution

2—Oxygenoid constitution

3—Hydrogenoid constitution

The Carbo-nitrogenoid constitution require higher potencies, the Oxygenoid constitution requires medium potencies and the Hydrogenoid constitution require lower potencies.

Potency and Nature of Disease

1—One-sided/far advanced/multi-systemic/organic disorders are required to be treated with low potencies. Giving high potencies in such cases can prove to be dangerous and can lead to unnecessary aggravations because of the lowered susceptibility of the individual in such cases.

2—Chronic diseases are to be treated with medium potencies as the susceptibility of patients suffering from chronic diseases becomes low with time.

3—Acute diseases come to the patients with great intensity and at that the susceptibility of the patient is high, so these cases require higher potencies.

Potency and Nature of Medicine

The nature of medicine also plays an important role in selection of potency.

1—Toxic products and Nosodes should be prescribed in high potencies.

2—Animal products, Mineral products and Sarcodes are better to be administered in medium potencies.

3—Plant products and Imponderbilia can be given to the individual in lower or medium potencies as per requirement of the case.

Potency and Pschological Constitution

Psychological constitution of the individual should also be considered while making prescription and selecting potency in the particular case. The psychological constitution can be of the following types:

1—Unstable constitution: These include nervous and bilious; melancholic temperament. These require lower potencies as giving high potencies in such cases can cause violent reaction and aggravation.

2—Stable constitution: These include sanguinine constitutions and they usually require medium potencies. And the other type under stable constitution is phlegmatic which are required to be administered higher potencies.

### OBJECT OF THE INVENTION

It is an object of the invention to provide a computer implemented method and system for facilitating to the medical experts in the selection of dose with correct potency which is based on Nigam's potency scale.

### BRIEF DESCRIPTION OF DRAWINGS

FIG. 1 shows a block diagram illustrating the system used for determining medical dose to patient based on Nigam's Potency scale.

### DETAILED DESCRIPTION OF THE INVENTION

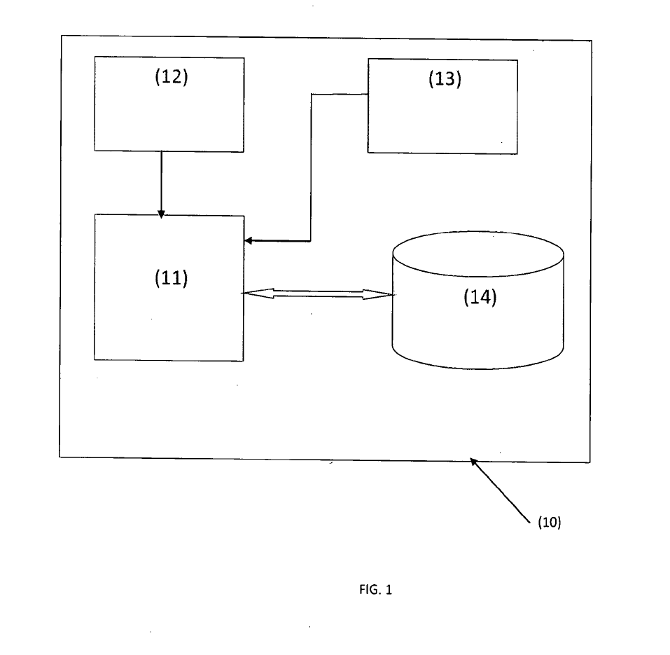

A computer system 10, suitable for implementing a method for determining medical dose to patient based on Nigam's Potency scale in accordance with the present invention as illustrated in FIG. 1. The said system consists a display unit (13) to display the result obtained after getting input through keyboard and processed by a processing unit. The input device (12) receives the case-taking information that the homeopath has elicited from the patient and may utilize a conventional keyboard and/or mouse. The display unit is used to display information to the homeopath and would typically be a CRT or printer. The processing unit (11) may contain a memory unit (14) such as a disk drive for holding data which may be used to store the instructions and also the information received from users of the system. A processing unit is provided for reading and writing of data from the database stored in memory, and for executing the operations based following Nigam's potency scale.

### Nigam's Potency Scale

| | | | | |
|---|---|---|---|---|
| PARAMETERS | 1 | 2 | 3 | MARKS |

| | | | |
|---|---|---|---|
| 1. Nature Of Medicine | Plant Products | Animal | Toxic |
| | Imponderbilia | Products | Products |
| | | Mineral | Nosodes |
| | | Products | |
| | | Sarcodes | |
| 2. Nature Of Disease | | | |
| Stage | One Sided | Chronic | Acute |
| | Far Advanced | Disease | Disease |
| | Multisystemic | | |
| | Disorder | | |
| Plane Of Disease | Body Matrix | Central | Mind |
| | | Controlling | |
| | | Axis | |
| | | Dynamic | |
| | | Plane | |
| 3. Susceptibility Of Patient | | | Sanguine |
| PhysicalConstitution | Phosphoric | Flouric | Carbonic |
| Biological Constitution | CarboNitrogenoid | Oxygenoid | Hydrogenoid |
| Psychological | Unstable | Stable | Stable |
| Constitution/Temperament | Nervous Bilious | Sanguine | Phlegmatic |
| | Melancholic | | |

Total Marks Scored

The details are as follows:

Nigam's Potency Scale

Nigam's potency scale works on a simple mathematical calculation. The computer implemented method takes in a set of numbers depending on the tabs a user clicks, adds them up and displays the result according to the range in which the number falls.

The scores range from 18 (lowest) to 54 (highest).

The division of scores and result is done as follows:
1) 18-30: Use low potency (1X/D1 to 6X/D6/3C)
2) 31-42: Use medium potency (4C TO 12C)
3) 43-46: Use high potency (30C)
4) 47-50: Use very high potency (200C)
5) 51-54: Use ultra-high potency (1M, 10M, 50M, 100M/CM; 1M=1000)

The judgment is done based on three conditions:
1) Nature of medicine
2) Nature of disease
3) Susceptibility of patient

Each of these rows has three columns and each column has a base value of 6, 12 and 18 respectively. Therefore, if the tab in the first column of each row is selected the result adds up to 18(6+6+6) which is the lowest possible and when the tab in the third column of each row is selected the result adds up to 54(18+18+18) which is the highest possible.

Now we see there are two sub-divisions in the second row ‘nature of disease’. In order to keep it bias free each tab in the first column of the second row is given a base value of 3. Therefore the two tabs in the first column add up to 6, hence, keeping the final result in balance. Same happens with column two and column three where the base values of each tab are set as 6 and 9 respectively, hence adding up to 12 and 18, therefore maintaining the balance.

Now we see the third row also has three subdivisions. Here also the bias removed through the same method by dividing the base value into three equal parts among the three different tabs in the same column. Therefore, each tab in the first column of the third row has a base value of 2, the tabs in the second column have 4 and finally the tabs in the third column have 6, hence maintaining the balance.

So basically the value distribution for each tab is as follows:

NIGAM'S POTENCY SCALE

| | | | |
|---|---|---|---|
| Nature of medicine | 6 | 12 | 18 |
| Nature of disease | | | |
| Subdivision1 | 3 | 6 | 9 |
| Subdivision2 | 3 | 6 | 9 |
| Susceptability of patient | | | |
| Subdivision1 | 2 | 4 | 6 |
| Subdivision2 | 2 | 4 | 6 |
| Subdivision2 | 2 | 4 | 6 |

Note:
It is compulsory to select at least one tab from each row.

The division of scores and result is done as follows:
1) 18-30: Use low potency (1X/D1 to 6X/D6/3C)
2) 31-42: Use medium potency (4C TO 12C)
3) 43-46: Use high potency (30C)
4) 47-50: Use very high potency (200C)
5) 51-54: Use ultra-high potency (1M, 10M, 50M, 100M/CM; 1M=1000)

I claim:

1. A computer implemented method for determining homoeopathic medicinal potency to patient based on Nigam's Potency Scale, the method comprising:
a) Receiving input data via input device based on patient symptoms;
b) Filling entries into Nigam's Potency Scale based on the nature of medicine, nature of disease and susceptibility of Patient;
c) Adding all the values entered in the form of numbers;
d) Displaying the information (potency) according to the range in which result of step c) falls.

2. A computer implemented method for determining homoeopathic medicinal potency to patient based on Nigam's Potency Scale as claimed in claim 1 wherein Nigam's Potency Scale represents nature of medicine, nature of disease and susceptibility of Patient by different rows and columns whereas every row has three columns and every column has original base value of 6,12 and 18 respectively.

3. A computer implemented method for determining homoeopathic medicinal potency to patient based on Nigam's Potency Scale as claimed in claim 1 wherein Nigam's Potency Scale scores range from 18(6+6+6) to 54(18+18+18).

4. A computer implemented method for determining homoeopathic medicinal potency to patient based on Nigam's Potency Scale as claimed in claims 1 and 2 wherein nature of disease further sub-divided and every sub-division gets base value which is exactly half of the original base value i.e. 3,6 and 9 respectively.

5. A computer implemented method for determining homoeopathic medicinal potency to patient based on Nigam's Potency Scale as claimed in claims 1 and 2 wherein susceptibility of Patient further subdivided into three sub-divisions and every sub-divisions gets base value which is exactly one third of original base value i.e. 2,3 and 6 respectively.

6. A computer implemented method for determining homoeopathic medicinal potency to patient based on Nigam's Potency Scale as claimed in claim 1 wherein potency is decided based on result obtained in step c) by following criteria:
1) If range of result lies between 18 to 30, Use low potency (1X/D1 to 6X/D6/3C)
If range of result lies between 31-42, Use medium potency (4C TO 12C)
If range of result lies between 43 to 46, Use high potency (30C),
If range of result lies between 47 to 50 Use very high potency (200C),
If range of result lies between 51-54, Use ultra-high potency (1M, 10M, 50M, 100M/CM; 1M=1000).

7. System for determining homoeopathic medicinal potency to patient based on Nigam's Potency scale, the said system (10) comprises:
a) Input device (12) for receiving information based on patient symptoms in the form of numbers and filing entries into Nigam's Potency scale which is based on medicine, nature of disease and susceptibility of Patient;
b) Memory unit (14) for storing instructions based on the Nigam's Potency scale;
c) Display unit (13) to display result based on the said Nigam's Potency scale;
d) Processing unit (11) configured to carried out addition of the all the entries made and result being displayed by the display unit according to claim 6.
